(19) United States
(12) Patent Application Publication
(10) Pub. No.: US 2005/0113768 A1
(43) Pub. Date: May 26, 2005

(54) Pain-alleviating injection needle

(76) Inventor: Carlos Patrickson, Edmonton (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/868,225

(22) Filed: Jun. 15, 2004

(30) Foreign Application Priority Data
Nov. 24, 2003 (CA) 2,448,541

(51) Int. Cl. A61M 5/32

(52) U.S. Cl. 604/272; 604/912

(57) ABSTRACT

A pain-alleviating injection needle includes a hollow body adapted to contain a liquid medication. A needle is provided having an attachment end and a piercing end. The attachment end is in fluid communication with the hollow body. A frangible membrane overlies the piercing end of the needle. The membrane is filled with a topical anesthetic. Upon the membrane being pressed against a selected injection site, the membrane ruptures releasing topical anesthetic onto the injection site, thereby numbing the injection site. Upon further pressure being exerted, the piercing end of the needle pierces the injection site.

### FIELD OF THE INVENTION

The present invention relates to a pain-alleviating injection needle and, in particular, an injection needle suitable for use by dentists.

### BACKGROUND OF THE INVENTION

In an attempt to overcome the aversion many people have to receiving injections with needles, some pain alleviating injection needles have been developed. U.S. Pat. No. 5,236,419 (Seney 1993) provides an example of such an injection needle. The Seney injection needle has an annular body with an axial central passage. The body is placed like a sheath over a needle, so that the needle is positioned within the central passage. The needle is movable between a retracted position retracted within the central passage and an extended position extending past the annular body. The annular body has hollow defining walls, that contain a substance, which can be frozen. In use, a patient's skin is numbed with the frozen annular body and then the injection needle is moved from the retracted position to the extended position, causing the injection needle to pierce the patient's skin.

Due to space limitations, the teachings of the Seney reference do not work particularly well in the field of dentistry.

### SUMMARY OF THE INVENTION

What is required is a pain-alleviating injection needle, which is better suited for use by dentists.

According to the present invention there is provided a pain-alleviating injection needle which includes a hollow body adapted to contain a liquid medication. A needle is provided having an attachment end and a piercing end. The attachment end is in fluid communication with the hollow body. A frangible membrane overlies the piercing end of the needle. The membrane is filled with a topical anesthetic. Upon the membrane being pressed against a selected injection site, the membrane ruptures releasing topical anesthetic onto the injection site, thereby numbing the injection site. Upon further pressure being exerted, the piercing end of the needle pierces the injection site.

### BRIEF DESCRIPTION OF THE DRAWINGS

These and other features of the invention will become more apparent from the following description in which reference is made to the appended drawings, the drawings are for the purpose of illustration only and are not intended to in any way limit the scope of the invention to the particular embodiment or embodiments shown, wherein:

FIG. 1 is a side view of the injection needle;

FIG. 2 is a end view of the injection needle;

FIG. 3 is a front view of the injection needle;

FIG. 4 is a perspective of the injection needle;

FIG. 5a is a side elevation view showing the end of the injection needle approaching the injection site;

FIG. 5b is a side elevation view showing the end of the injection needle with the frangible membrane ruptured; and

FIG. 5c is a side elevation view showing the needle being injected.

### DETAILED DESCRIPTION OF THE PREFERRED EMBODIMENT

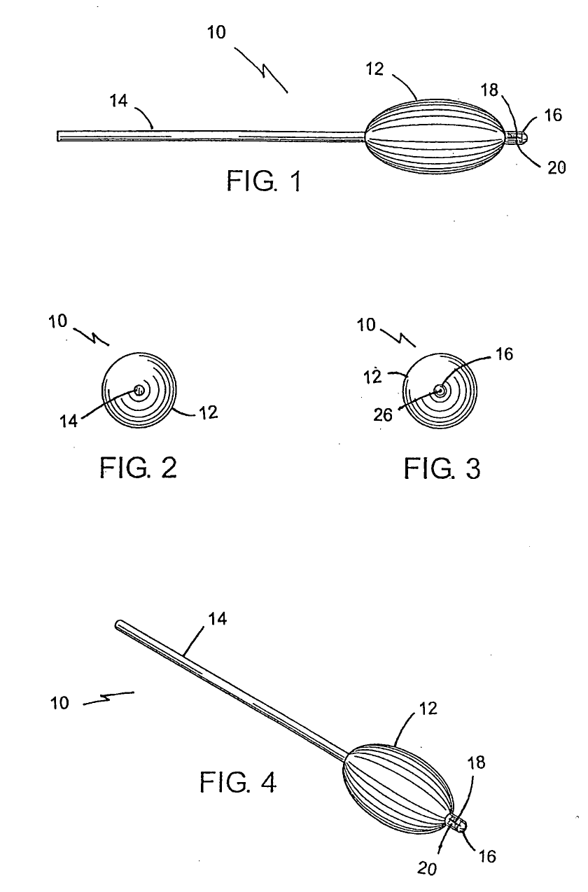

The preferred embodiment, a pain-alleviating injection needle generally identified by reference numeral 10, will now be described with reference to FIGS. 1 through 5c.

Structure and Relationship of Parts:

Referring to FIGS. 1, 2, 3, and 4 the pain relieving injection needle 10 includes a hollow body 12 in the form of a bladder, which is adapted to contain a liquid medication or anaesthetic. A handle 14 is provided to ensure that hollow body 12 is not lost within the patient's mouth. A needle 18 is provided which has an attachment end 24 and a piercing end 26. Attachment end 24 is in fluid communication with hollow body 12. A frangible membrane 16 overlies piercing end 26 of needle 18. Membrane 16 is adapted to rupture, when pressed against a selected injection site 28. Membrane 16 is filled with a topical anaesthetic 20. When membrane 16 ruptures, topical anaesthetic 20 is released onto injection site 28, thereby numbing injection site 28. As further pressure is exerted, piercing end 26 of needle 18 then pierces injection site 28. It is preferred that hollow body 12 be coated with a candy flavouring, which is released when exposed to moisture in a patient's mouth.

Operation:

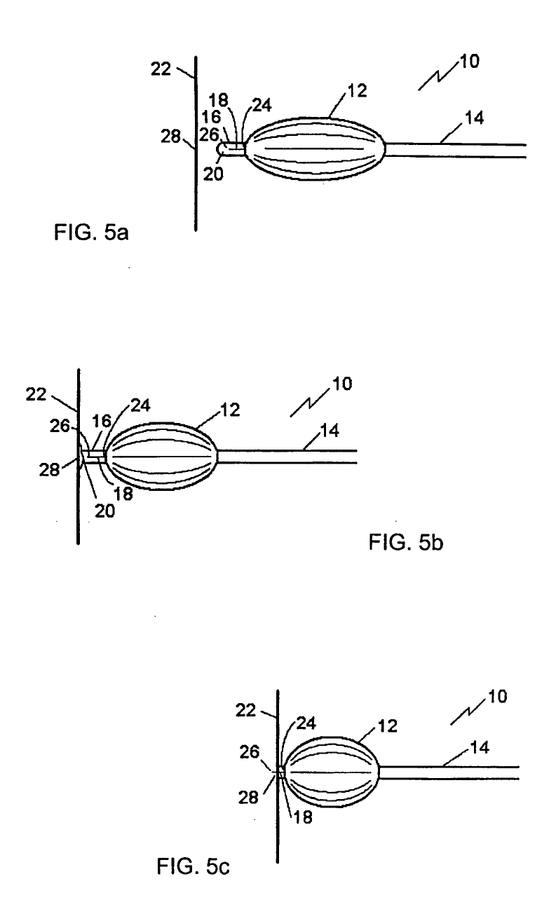

Referring to FIG. 5a, the injection needle 10 is shown approaching the skin 22 of a patient. In FIG. 5b, pressure is applied such that the frangible membrane 16 is ruptured, and topical anaesthetic 20 is released to numb injection site 28. In FIG. 5c, further pressure is exerted, and the piercing end 26 of needle 18 pierces injection site 28. The liquid medication or anaesthetic in the hollow body 12 may then be injected by manually squeezing the hollow body 12.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent to one skilled in the art that modifications may be made to the illustrated embodiment without departing from the spirit and scope of the invention as hereinafter defined in the claims.

1. A pain-alleviating injection needle, comprising:
a hollow body adapted to contain a liquid medication;
a needle having an attachment end and a piercing end, the attachment end being in fluid communication with the hollow body; and
a frangible membrane overlying the piercing end of the needle, the membrane being filled with a topical anesthetic, such that upon the membrane being pressed against a selected injection site, the membrane ruptures releasing topical anesthetic onto the injection site, thereby numbing the injection site, and upon further pressure being exerted, the piercing end of the needle pierces the injection site.

2. The pain-alleviating injection needle as defined in claim 1, wherein the body is a bladder from which liquid medication is manually squeezed.

3. The pain-alleviating injection needle as defined in claim 2, wherein the body has a handle.

4. The pain alleviating injection needle as defined in claim 1, wherein the body has candy flavouring, which is released when exposed to moisture in a patient's mouth.

5. A pain-alleviating injection needle, comprising:
a hollow bladder adapted to contain a liquid medication;
a candy coating on the bladder providing a candy flavouring which is released when exposed to moisture in a patient's mouth;
a needle having an attachment end and a piercing end, the attachment end being in fluid communication with the bladder, such that when the bladder is manually squeezed the liquid medication passes from the attachment end to the piercing end of the needle;
a frangible membrane overlying the piercing end of the needle, the membrane being filled with a topical anesthetic, such that upon the membrane being pressed against a selected injection site, the membrane ruptures releasing topical anesthetic onto the injection site, thereby numbing the injection site, and upon further pressure being exerted, the piercing end of the needle pierces the injection site; and
a handle secured to the bladder, such that by gripping and exerting a force upon the handle the bladder is removed from a patient's mouth.
